(19) United States
(12) Patent Application Publication
(10) Pub. No.: US 2026/0117187 A1
(43) Pub. Date: Apr. 30, 2026

(54) LIPID NANOPARTICLES FOR BIOPRODUCTION

(71) Applicant: Worcester Polytechnic Institute, Worcester, MA (US)

(72) Inventors: Daniel Zimmer, Worcester, MA (US); Christina M. Bailey-Hytholt, Worcester, MA (US); Luke R. Perreault, Worcester, MA (US); Glenn R. Gaudette, Holden, MA (US)

(21) Appl. No.: 19/370,173

(22) Filed: Oct. 27, 2025

Related U.S. Application Data
(60) Provisional application No. 63/711,991, filed on Oct. 25, 2024.

(51) Int. Cl. C12N 5/077; C07K 14/50; C12N 15/85; C12N 15/88

(52) U.S. Cl. CPC C12N 5/0659; C07K 14/503; C12N 15/85; C12N 15/88; C12N 2510/00; C12N 2830/003; C12N 2830/005

(57) ABSTRACT

A nonviral gene therapy delivers DNA that allows cells to produce proteins such as their own growth factors in culture media. Lipid nanoparticles provide an effective delivery mechanism into a target cell. By encapsulating plasmid DNA encoding the growth factors with inducible promoters into lipid nanoparticles (LNPs), target cells create their own growth factors reducing a need for expensive growth factors from external sources. DNA expression lasts for several days reducing the amount of reagent needed. The plasmid DNA is more stable than mRNA and proteins, therefore reducing logistical challenges.

### RELATED APPLICATIONS

This patent application claims the benefit under 35 U.S.C. § 119 (e) of U.S. Provisional Patent App. No. 63/711,991, filed Oct. 25, 2024, entitled “AUTOCRINE GROWTH FACTOR PRODUCTION,” incorporated herein by reference in entirety.

### BACKGROUND

A continually rising world population has induced an unsustainable demand for growth on conventional chemical and protein production, which is already hampered by the proliferation of climate change and increasingly strained arable land and fresh water. Bioreactor based manufacturing offers a method to alleviate these concerns by shifting the burden of chemical and protein production to a cell-based bioreactor production model. However, it is limited in its scalability by the need for high volumes of expensive cell culture growth factors and biomolecules. Culture media, specifically the growth factors, are a significant cost for cellular agriculture (CellAG) and biomanufacturing. Reduction in the cost burden of biomanufacturing allows sustainable biomanufacturing for food sources, pharmaceuticals and other bioproduction.

### SUMMARY

A nonviral gene therapy delivers DNA that allows cells to produce proteins such as their own growth factors in culture media. Lipid nanoparticles provide an effective delivery mechanism into a target cell. By encapsulating plasmid DNA encoding the growth factors with inducible promoters into lipid nanoparticles (LNPs), target cells create their own growth factors, reducing a need for expensive growth factors from external sources. DNA expression lasts for several days, reducing the amount of reagent needed. The plasmid DNA is more stable than mRNA and proteins, therefore reducing logistical challenges.

Configurations herein are based, in part, on the observation that agricultural and bioproduction have potential to satisfy demand for agricultural and biological products, such as plant and protein based materials capable of supplying food source channels of commerce. Unfortunately, conventional approaches to bioproduction suffer from the shortcoming that growth factors, often employed for cell engineering and tissue growth, impose a substantial expense on a scalable approach. Accordingly, configurations herein substantially overcome the shortcomings of conventional approaches by providing a plasmid DNA encoding of growth factors using lipid nanoparticles. Lipid nanoparticles provide an effectively delivery mechanism into a cell for implantation of DNA or other nucleic acids for self-expression of growth factors within the cell.

Plasmid DNA is a special type of DNA found mainly in bacteria that exists as a small, circular piece separate from the main chromosome. This is typically a small, circular DNA molecules found outside a main chromosome in bacteria and some other microbes, thus differs from large, linear chromosomes that carry most of an organism's genetic information.

More specifically, in conventional approaches, muscle progenitor cells such as bovine satellite cells (BSCs) must be cultured with growth factors such as fibroblast growth factor 2 (FGF2), vascular endothelial growth factor (VEGF), and others to maintain proliferation. This is a major cost burden compromising scale-up of biomanufacturing and CellAg.

In a particular configuration, a method for forming engineered cells includes identifying a target cell having a bioproduction value, and generating a nucleotide sequence configured for expressing a growth factor within the target cell. The nucleotide sequence is introduced into the target cell for bioproduction resulting from growth factor expression in the target cell. In a particular configuration, this involves encapsulating a plasmid DNA encoding of a growth factors with an inducible promoter, such as bovine satellite cells (BSCs) into lipid nanoparticles (LNPs). The LNPs are then introduced into a muscle progenitor cell to induce transient expression of FGF2 (fibroblast growth factor 2) for controlling growth factor expression and production by satellite cells. An inducible promoter allows for selectively controlling the expression of the FGF2.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a lipid delivery mechanism as disclosed herein;

FIGS. 2A-2C are fluorescence measurements for cells of the lipid delivery mechanism of FIG. 1;

FIGS. 3A-3C show flow cytometry results quantifying cow (bovine satellite muscle cells) and quail cells (QM7) treated with GFP mRNA; and

FIGS. 4A and 4B show hydrodynamic diameter for the LNP formulations used to treat cells of FIGS. 3A and 3B, respectively.

### DETAILED DESCRIPTION

The description below presents an example of genetically modifying BSCs to self-produce FGF2 to help maintain stemness, reducing costs by eliminating the need for exogenous FGF2 in culture media. Configurations herein employ the use of lipid nanoparticle (LNP)-delivered plasmid DNA (pDNA) to induce transient expression of FGF2, as a method to control growth factor expression and production by satellite cells, and potentially enable this type of engineered autocrine signaling in unmodified primary cells. This represents an economically viable approach as (1) it is based upon established LNP-delivery protocols used therapeutically in industry, (2) allows growth factor expression to be “turned on or off” through the use of a Tet (Tetracycline-inducible), GAL4/UAS (Yeast-derived), or lac repressor and/or cessation of LNP treatments supporting strong control of cell state, and (3) does not permanently alter cellular gene expression, which may support consumer and regulatory acceptance. The lac repressor system itself does not drive cell growth, but rather is a gene-expression control system originally from bacteria. Configurations herein demonstrate this by developing an LNP-based system with controllable autocrine signaling for primary bovine satellite cells. Specifically, disclosed configurations propose that pDNA-induced FGF2 (hereafter referred to as pFGF2) expression will maintain BSC stemness consistent with exogenous FGF2 supplementation in media. It is expected that cells will differentiate upon treatment termination, as pFGF2 expression decreases. The disclosed examples employ bovine cells as a non-limiting example, most notably due to its potential value as a food product, as the disclosed nanoparticle introduction may be employed with any suitable cell system or organism.

As employed herein, a lipid nanoparticle (LNP) is a small, spherical delivery vehicle made from fats (lipids) that can carry substances such as drugs, RNA, or other therapeutic molecules into cells. The LNP shell is formed of lipids that are similar to the fats in human cell membranes, while encapsulated it can protect fragile cargo such as mRNA from being broken down. LNP particles are designed to fuse with a cell's membrane, allowing the cargo, such as mRNA (messenger RNA), siRNA (short interfering RNA) or pDNA, to enter the cell.

One of the potential avenues of disclosed configuration addresses bioprocess strategies to facilitate affordable cell and protein production. Conventional approaches exhibit a high cost-to-yield ratio that curtails market growth for tissue engineered products. Expansive bioproduction of cells and proteins carries substantial logistical challenges, such as regulation of proliferation, differentiation and O2/nutrient management all represent needs to be considered. Further, the use of techniques and materials standard in the pharmaceutical industry is prohibitively expensive.

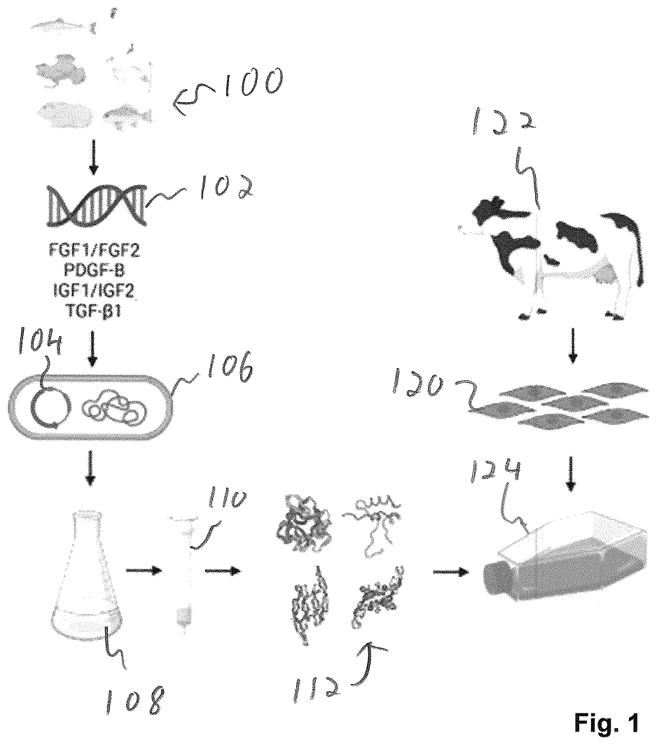

FIG. 1 is a context diagram of a lipid delivery mechanism as disclosed herein. In examples herein, the use of animal-derived products undesirable from welfare, regulatory, and cost perspectives. For example, fetal bovine serum is rather price volatile, however synthetic alternatives may present lower, more stable media costs. While animal-free alternative serum formulations may be considered, these remain less effective than conventional media. Referring to FIG. 1, a growth factor is derived from any suitable source 100, such as laboratory, synthetic or animal sources. A nucleotide sequence 102 is generated for expressing the growth factor within the target cell including the growth factor. As defined herein, a growth factor is a signaling molecule that cells release to tell other cells to grow, divide, survive, or specialize. The growth factor provides the chemical “instructions” that control how tissues develop, repair themselves, and respond to their environment. Example growth factors include FGF1, FGF2 (fibroblast growth factor 1, 2), PDGF-B (Platelet-Derived Growth Factor-B), IGF1, IGF2 (Insulin-like Growth Factor 1, 2), or a TGF (Transforming Growth Factor). Other suitable growth factors may be included.

In general, Fibroblast Growth Factors (FGFs) are a family of signaling proteins that guide cells on how to grow, survive, and develop. They are especially important during early development and throughout life for maintaining and repairing tissues. FGFs, such as FGF2, bind to specific receptors on cell surfaces called FGF receptors (FGFRs), and activate signaling pathways inside the cell that influence growth, movement, and differentiation.

In the example configuration, the nucleotide sequence is formed as a plasmid DNA 104, and the plasmid DNA encapsulated for encoding the growth factors with inducible promoters into a lipid nanoparticle (LNP) 106. In general, the growth factor binds to a specific receptor on another cell's surface, i.e. the target cell, and triggers signals inside the cell such that the target cell changes its behavior, often for proliferation, movement, or differentiation.

In the configurations herein, the inducible promoters are segments of DNA that control when a gene is turned on, which activate in response to a specific signal. Such promoters perform similar to a switch responsive to certain predetermined conditions. A promoter is effectively an “on/off control” region before a gene, and is “Inducible,” meaning the promoter stays off by default and turns on when exposed to an inducer or vice versa. These promoters can be chemical in nature, or use a physical signal such as light or temperature.

A particular beneficial feature is to invoke a nucleotide sequence configured to express an autocrine growth factor. This induces autocrine signaling such that the cell secretes a growth factor, the same cell has the corresponding receptor on its own surface, and the growth factor then binds the receptor. Thus, the cell activates internal growth and survival pathways, and can create a self-reinforcing loop such that the cell continues to produce its own growth factor, relieving the need for costly introduction of additional growth factor. Other suitable growth factors and/or culture media components may also be expressed based on generating an appropriate nucleotide sequence, or proteins having a complementary or symbiotic effect with the cell or expressed growth factors. In summary, any suitable amino acid or nucleotide for which the plasmid DNA can be encoded to express may be employed, provided it binds with the target cell.

Introducing the nucleotide sequence 102 into the target cell involves defining a delivery mechanism based on at least one of a lipid, ionizable lipid, polymer, and/or nanoparticle, such that the delivery mechanism is based on transfecting the cell or protecting the nucleotide sequence from degradation. The delivery mechanism is typically based on at least one of a lipid, ionizable lipid, polymer, and/or nanoparticle, the delivery mechanism based on transfecting the cell or protecting the nucleotide sequence from degradation. A particular configuration employs a delivery mechanism 108 including one or more of ionizable lipid, phospholipid such as PC (phosphatidylcholine) or PE (phosphatidylethanolamine), cholesterol, and PEGlyated lipid.

By way of background, Phospholipids are a large category of molecules that make up cell membranes, including PC (phosphatidylcholine), PE (phosphatidylethanolamine) PI (phosphatidylinositol), PS (phosphatidylserine), and often combined with SPH (Sphingosine) as a backbone. Lipid nanoparticle (LNP)-delivered plasmid DNA (pDNA) induces transient expression of FGF2. For example, a conventional LNP for mRNA delivery may contain:
An ionizable lipid (for binding and releasing RNA);
A phospholipid (often DSPC or DOPE);
Cholesterol (for membrane stability);
A PEGylated lipid (improves circulation and prevents aggregation).
   Although the ionizable lipid is often the novel component in RNA delivery, the phospholipid, Cholesterol, and PEGylated lipid provide a significant supporting component that helps the particle form correctly and deliver its payload effectively.

The plasmid DNA is prepared for a particular target cell having a bioproduction value, such as for protein, cell or other biomanufacturing application. A particularly beneficial usage is for protein tissue for human consumption, i.e. cultured media for meat. In an example configuration, the target cells are muscle progenitor cells such as bovine satellite cells (BSCs) 120 or other eukaryotic cell, derived from natural stock 122 or other suitable source, or prokaryotic cell including bacteria such as Escherichia coli. It should be noted that the use of BSCs as target cells does not impose a need for fetal bovine serum, which the present approach seeks to avoid.

The delivery mechanism includes a physical delivery 110, in which LNPs are combined with culture media through a pipette, syringe or other suitable introduction, often in a containment 124, for combining the LNPs 106, and related delivery media 112 with the target cells 120. Upon LNP fusion with the cell, the payload is internalized, such that the physical delivery provides introduction of the nucleotide sequence 102 into the target cell 120 for bioproduction resulting from growth factor expression in the target cell.

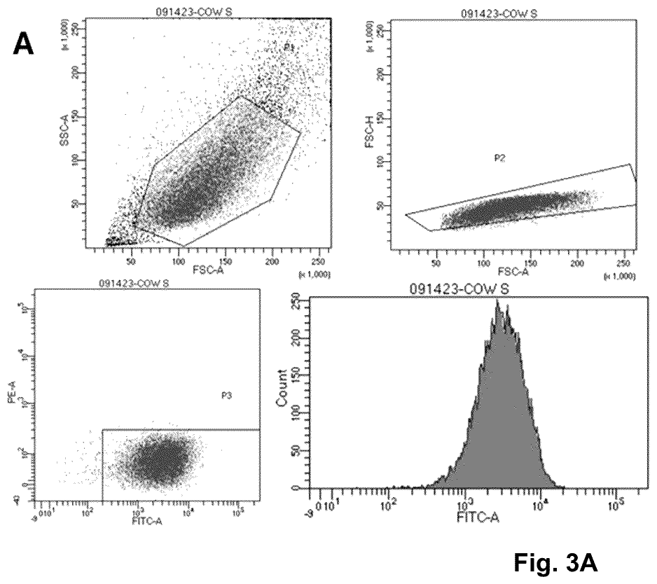

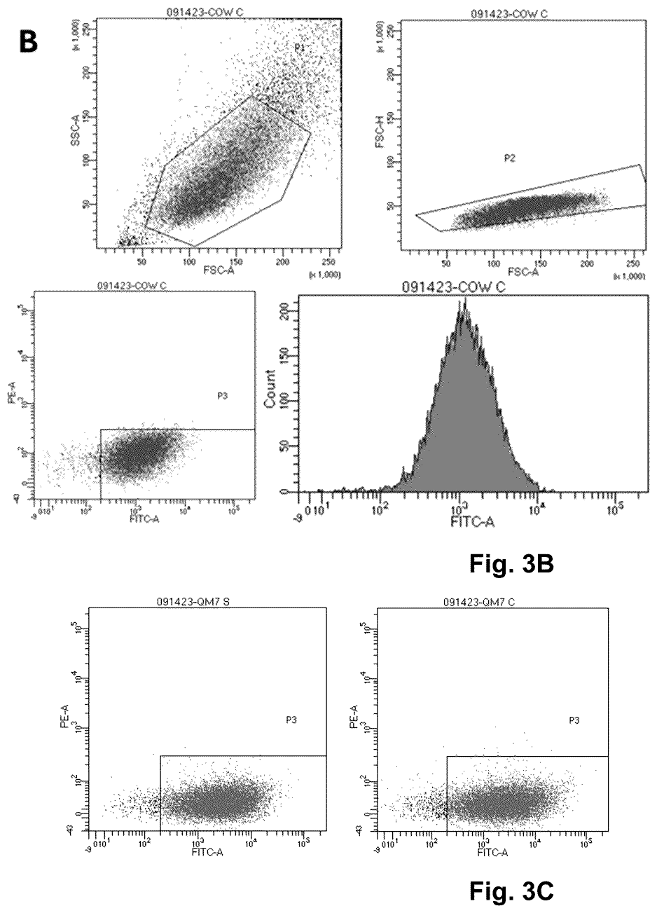

Example configurations have formulated LNPs utilizing promising ionizable lipids for the delivery of nucleic acids to bovine satellite stem cells, a notoriously difficult cell type to transfect. In conventional approaches, the design of nonviral gene therapies is synonymous with safety, lipids are designed to have low cytotoxicity, DNA is avoided for the risk of incorporation into the genome, and purity of components is paramount. This design criteria drives up costs, but these risks are less prominent in cellular agriculture allowing for significant cost reduction. Because of the importance of LNP formulation on delivery efficacy, initial configurations performed an assay delivering a GFP (Green Fluorescent Protein)-expressing pDNA to primary bovine satellite cells within LNP formulations containing SM-102 and C12-200 ionizable lipids, discussed further below with respect to FIGS. 3A,B and 4A,B. High GFP expression was observed using both vehicles, but with greater fluorescence intensity using SM-102 LNPs. Therefore, it was concluded that pDNA can be effectively delivered into primary bovine satellite cells with LNPs.

Accordingly, configurations herein seek to identify and develop an optimal LNP synthesis protocol for pDNA delivery to BSCs. This involves comparison of lipids used in FDA-approved therapeutic formulations, such as SM-102 (from Spikevax), with alternative lipids such as those having higher transfection efficiency, such as C12-200. LNP formulations will be designed for highly efficient delivery of pDNA, assessed by inducing green fluorescent protein (GFP) expression in cells while balancing the cost parity of alternative media formulations. GFP expression will be used as a metric of delivery and expression efficacy, measured via flow cytometry.

Disclosed configurations also induce and quantify production of pFGF2 by BSCs and determine efficiency in maintaining BSC proliferation versus exogenous FGF2 supplementation in culture media. Use of pFGF2 expression must both produce FGF2 and support proliferation at a consistent level compared to conventional FGF2 supplementation. This includes evaluating pFGF2 production using an enzyme-linked immunosorbent assay (ELISA) of culture media, monitor stemness through immunofluorescence quantification of Pax7, and proliferative cells through quantification of Ki67 (a protein marker of cell proliferation). Various other culture media may be induced based on the plasmid DNA and media it is encoded to express, such as stem cell media with extra growth factors to keep them undifferentiated, serum-free media designed to reduce immune reactions or variability, and selective media to grow only cells with a specific gene or trait. For example, biologically active proteins present in culture media viable for production using the disclosed approach include Albumin, Transferrin, Insulin, Fibronectin, Vitronectin, Fetuin-A, Growth factors including VEGF (Vascular Endothelial Growth Factor), EGF (Epidermal Growth Factor), FGFn (Fibroblast Growth Factor n), IGF (Insulin-Like Growth Factor 1, 2), PDGF (Platelet-Derived Growth Factor), and TGF-β (Transforming Growth Factor-beta), and Protease inhibitors.

In a particular example, for cow cells transfected with GFP LNPs the following expression was observed: 29 F.U. in 98.5%, 2772 F.U. in 99.3% percent of the population, and 1113 F.U. in 96.5% percent of the population where observed for negative control, SM-102, and C12-200 LNPs respectively. For quail cells transfected with GFP LNPs the following expression was observed: 17 F.U. in 99.8% percent of the population, 2503 F.U. in 99.7% percent of the population, and 2176 F.U. in 99.7% percent of the population for negative control, SM-102, and C12-200 LNPs respectively. Finally, for human fibroblasts transfected with GFP LNPs the following expression was observed: 47 F.U. in 99.1% percent of the population, 180 F.U. in 99.9% of the population, and 226 F.U. in 99.9% where observed for negative control, SM-102, and C12-200 LNPs respectively. While C12-200 has been shown to have excellent delivery performance in humans, however we saw that it was underperformed by SM-102 (the ionizable lipid used in the Moderna vaccine). This difference could be due to the molar ratio of ionizable lipids used or it could be due to differences in drug delivery to animal cells.

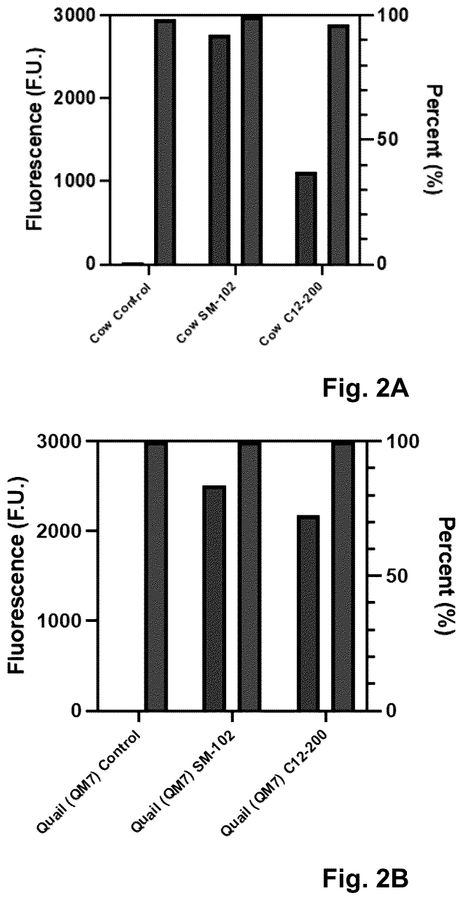

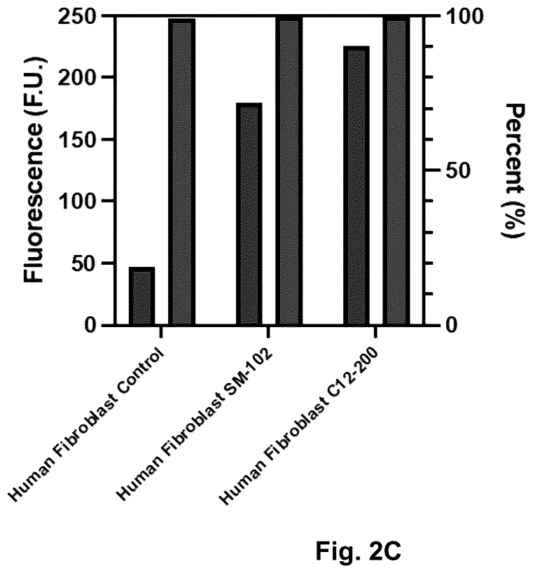

FIGS. 2A-2C show relevant results. On the left y-axis of each graph is the mean fluorescence units per cell and on the right y-axis is the percent of cells above the fluorescent cutoff. FIG. 2A depicts cow (bovine satellite muscle cells); FIG. 2B shows immortalized quail cell line QM7; and FIG. 2C is a human fibroblast cell line, each graph is looking at negative control, SM-102, and C12-200 as ionizable lipids for the LNPs, and for each cell line the left entry is a median fluorescence and the right entry is the percent of population.

LNP therapeutics utilize an ionizable lipid to load nucleic acids. The ionizable lipid typically has a pKa below 7, is mixed with a low pH solution resulting in protonation of the amine and complexation with negatively charged phosphates of nucleic acids. Because the amine (N) and phosphates (P) are so important, the N/P ratio is a defining characteristic during formulation. After LNP formation, a buffer exchange is performed, resulting in a neutrally charged particle and decreased cytotoxicity upon administration compared to cationic particles. The structure of the LNP remains intact with the hydrophobicity of the carbon chains. Upon administration, the LNP shields the encapsulated nucleic acid from degradation following administration and helps with cellular delivery by promoting endocytosis.

FGF2 is a significant component of culture media, often supplemented with fetal bovine serum (FBS) in conventional approaches. Unfortunately, sources of growth factors are expensive and FGF2 has a limited shelf life meaning that for applications such as cellular agriculture. As mentioned in the discussion on exosomes, insufficient exposure to growth factors will cause differentiation of cells. In the instance of cellular agriculture, muscle stem cells (satellite cells) differentiate into myocytes, once differentiated myocytes enter a quiescent state. Because of this the cells have to be kept in their stem state during expansion using growth factors the entire time. This is a major cost burden on the development of cellular agriculture causing academics and companies to actively seek solutions.

FIGS. 3A-3C show flow cytometry results quantifying fluorescence of cow (bovine satellite muscle cells) and quail cells (QM7) treated with GFP mRNA utilizing (A) SM-102 LNPs and (B) C12-200 LNPs. FIG. 3A shows an SM-102 average 131.033 nm with 0.254 PDI, 8.7 mV and EE 99.8%. FIG. 3B shows a C12-200 average 211.9 nm with 0.164 PDI, 18 mV and EE 101.4%. In FIG. 3C, S is for SM-102 ionizable lipids and C is for C12-200.

A particular use of bovine satellite cells facilitates subsequent cell activity and growth. Bovine myoblasts are muscle precursor cells derived from bovine satellite cells. They are crucial in the development, growth, and repair of skeletal muscle tissue in cattle. Myoblasts originate from satellite cells, a type of stem cell found in skeletal muscle. When activated, satellite cells proliferate and differentiate into myoblasts (myoblasts), which facilitate muscle growth and regeneration. Myoblasts can fuse to form muscle fibers (myofibers), contributing to muscle hypertrophy (growth) or repair following injury.

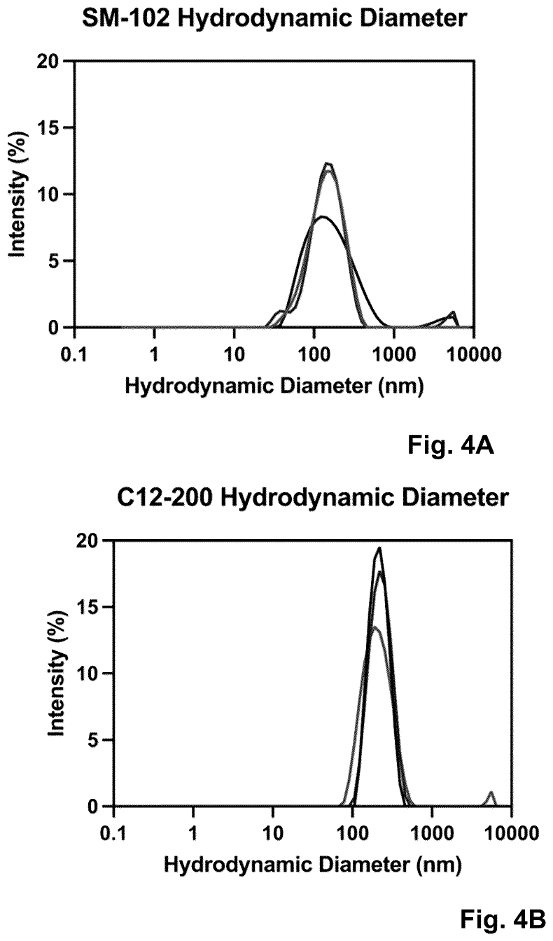

FIGS. 4A and 4B show hydrodynamic diameter and polydispersity of the LNPs used to transfect the cells in FIGS. 3A and 3B, respectively.

TABLE I

| Component | Percentage of Composition |
|---|---|
| Ionizable lipid | 50 ± 50 |
| Cholesterol | 35 ± 50 |
| PEGylated Lipid | 1.5 ± 10 |
| Helper lipid | 10 ± 50 |

In a further example, referring to Table I, compositions vary significantly with ionizable lipid type. For example, C12-200:DSPC:Cholesterol:PEG2000-PE at a molar ratios of 35, 10, 53.5 and 1.5%, respectively. Total lipid concentrations used in this study ranged from 5 to 35 mM. The LNPs were formulated with a target nitrogen to phosphate (N/P) ratios of 25. Typically, N/P ratios range for 3-8 for single amine lipids, in comparison C12-200 has five amines making this a similar molar ratio of lipids to phosphates. Nucleic acids were diluted in citrate buffer at a concentration resulting in the appropriate N/P ratio. As used herein, a “cell” refers to a biological cell. Some non-limiting examples include: a prokaryotic cell, eukaryotic cell, a bacterial cell, an archaea cell, a cell of a single-cell eukaryotic organism, a protozoa cell, a cell from a plant, an algal cell, a fungal cell, a fungal protoplast cell, an animal cell, and the like. Sometimes a cell is not originating from a natural organism, e.g., a cell can be a synthetically made, sometimes termed an artificial cell.

Although various features of the disclosed approach may be described in the context of an example configuration, the features can also be provided separately or in any suitable combination. Conversely, although the disclosure may be described herein in the context of separate embodiments for clarity, various aspects and embodiments can be implemented in a single embodiment.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for forming engineered cells, comprising:
delivering a nucleotide sequence configured for expressing a growth factor or other culture media component within a target cell; and
allowing for an expression of the growth factor or other media component in the target cell for bioproduction.

2. The method of claim 1, wherein the nucleotide sequence is a plasmid DNA, further comprising encapsulating the plasmid DNA encoding for one or more growth factors or other culture media components.

3. The method of claim 1, wherein introducing the nucleotide sequence into the target cell further comprises a delivery mechanism based on at least one of a lipid, ionizable lipid, polymer, and/or nanoparticle, the delivery mechanism based on transfecting the cell or protecting the nucleotide sequence from degradation.

4. The method of claim 3, wherein the delivery mechanism further comprises one or more of Ionizable lipid, phospholipid, Cholesterol, and/or PEGylated lipid.

5. The method of claim 2, wherein the lipid nanoparticles are formulated with one or more inducible promoters within the lipid nanoparticles (LNPs).

6. The method of claim 1, wherein the target cell is a eukaryotic cell.

7. The method of claim 1, wherein the target cell is a prokaryotic cell.

8. The method of claim 1, wherein the nucleotide sequence is a DNA sequence.

9. The method of claim 1, wherein the expressed growth factor is one or more autocrine growth factor such as FGF1, FGF2 (fibroblast growth factor 1, 2), PDGF-B (Platelet-Derived Growth Factor-B), IGF1, IGF2 (Insulin-like Growth Factor 1, 2), or a TGF (Transforming Growth Factor).

10. The method of claim 1, wherein the expressed media component is a protein having a complementary or symbiotic effect when added to the culture media.

11. The method of claim 1, wherein the nucleotide sequence is configured to express an autocrine growth factor without an inducible promoter.

12. The method of claim 1, wherein the nucleotide sequence is responsive to an inducible promotor for controlling grown factor generation.

13. The method of claim 1, wherein the nucleotide sequence contains a Tet (Tetracycline-inducible), GAL4/UAS (Yeast-derived), or lac repressor and/or uses cessation of LNP treatments supporting strong control of cell state.

14. A compound resulting from:
identifying a biomanufactured cell for bioproduction;
encapsulating plasmid DNA encoding of a growth factor with an inducible promoter into lipid nanoparticles (LNPs);
introducing lipid nanoparticle (LNP)-delivered plasmid DNA (pDNA) into the biomanufactured cell to induce transient expression of FGF2 (fibroblast growth factor 2) for controlling growth factor expression and production by satellite cells; and
selectively controlling the expression of the FGF2 based on the inducible promoter.

15. The compound of claim 14, wherein the biomanufactured cells are muscle progenitor cells.

16. The compound of claim 14, wherein the muscle progenitor cells are bovine satellite cells (BSCs).

17. A method for inducing cell growth, comprising:
selecting growth factors for a biomanufactured product;
plasmid DNA encoding the growth factors with inducible promoters; and
encapsulating the encoded growth factors into lipid nanoparticles (LNPs).

18. The method of claim 17, further comprising introducing the encoded growth factors into a cell sample for inducing the cell sample to generate additional growth factors.

19. The method of claim 17, further comprising employing the LNPs as a delivery vehicle for the growth factor.

20. The method of claim 1, further comprising:
selecting the target cell based on a bioproduction value;
generating a plasmid DNA configured for expressing the growth factor; and
delivering the plasmid DNA to the target cell via a lipid nanoparticle for inducing the cell to express the growth factor.
